(19) United States
(12) Patent Application Publication
(10) Pub. No.: US 2026/0345196 A1
(43) Pub. Date: Nov. 5, 2026

(54) DEVICES AND METHODS FOR EPICARDIAL ACCESS

(71) Applicants: Jinhoon PARK, Mason, OH (US); Riley MARANGI, Mason, OH (US); Danielle REDINBAUGH, Mason, OH (US); Brian FLORES, Mason, OH (US); Matt WINKLER, Mason, OH (US); Ethan WAYMAN, Mason, OH (US); Michael J. BANCHIERI, Mason, OH (US); Tamer IBRAHIM, Mason, OH (US); Ara STEPHANIAN, Mason, OH (US); Robert L. CLARK, III, Mason, OH (US); Zack BORGLIN, Mason, OH (US); Kenneth L. MILLER, Mason, OH (US); Alex KOGAN, Mason, OH (US)

(72) Inventors: Jinhoon PARK, Mason, OH (US); Riley MARANGI, Mason, OH (US); Danielle REDINBAUGH, Mason, OH (US); Brian FLORES, Mason, OH (US); Matt WINKLER, Mason, OH (US); Ethan WAYMAN, Mason, OH (US); Michael J. BANCHIERI, Mason, OH (US); Tamer IBRAHIM, Mason, OH (US); Ara STEPHANIAN, Mason, OH (US); Robert L. CLARK, III, Mason, OH (US); Zack BORGLIN, Mason, OH (US); Kenneth L. MILLER, Mason, OH (US); Alex KOGAN, Mason, OH (US)

(21) Appl. No.: 19/705,507

(22) Filed: Jun. 11, 2026

Related U.S. Application Data
(63) Continuation-in-part of application No. PCT/US2024/060154, filed on Dec. 13, 2024.
(60) Provisional application No. 63/610,921, filed on Dec. 15, 2023.

(51) Int. Cl. A61B 17/34; A61B 1/00; A61B 17/00; A61B 18/00; A61B 18/14; A61B 90/00

(52) U.S. Cl. CPC A61B 17/34; A61B 1/00147; A61B 18/1477; A61B 90/39; A61B 2017/00566; A61B 2018/00184; A61B 2018/00363; A61B 2018/00601; A61B 2018/00982; A61B 2090/3966

(57) ABSTRACT

Disclosed herein are systems and devices for accessing an epicardial space of a patient. The system can comprise an elongated body comprising a proximal end, a distal end, and a lumen therethrough. The system can further comprise a distal tip at the distal end of the elongated body, wherein the distal tip further comprises a distal window. The system can further comprise an endoscope positioned through the lumen, wherein an endoscope tip is positioned within the distal window, a vacuum source in fluid communication with the lumen, wherein the vacuum source is configured to draw tissue into the lumen through the distal window, a needle advanceable through the lumen, wherein the needle is configured to be advanced through the tissue, wherein the needle remains within the distal window when advanced through the tissue, and a guidewire advanceable through the needle, the guidewire being configured to advance through the tissue.

### CROSS-REFERENCE TO RELATED APPLICATIONS

This application is a continuation-in-part of International Application No. PCT/US 2024/060154 filed Dec. 13, 2024, which claims benefit of U.S. Provisional Application No. 63/610,921 filed Dec. 15, 2023, the entirety of which is incorporated by reference.

### FIELD OF TECHNOLOGY

The present disclosure relates generally to the field of epicardial access, and more specifically, accessing the pericardium safely and repeatably.

### BACKGROUND

There are several procedures currently being done in the epicardial space by electrophysiologists using a percutaneous approach. The state of the art for access involves using a needle and fluoroscopic guidance to access the pericardium and place a guidewire. This approach carries a substantial level of risk of puncture of unintended tissue (right ventricle, liver, diaphragm) with the needle that it has proven to be a barrier to entry for some electrophysiologists.

Further, the sharpness of the needle can shave material off the guidewire during use, which can lead to particulate being left within the anatomy. Existing devices either do not provide for visualization or suction, or advance through the tissue with a needle.

Therefore, there remains a need for methods and devices for using visualization and vacuum to draw the pericardium into the device and visualize insertion of the needle into the pericardium while eliminating the possibility of inadvertent puncture and particulate remains after use.

### SUMMARY

In some variations, the techniques described herein relate to a device for accessing an epicardial space of a patient, the device including: an elongated body including a proximal end, a distal end, and a lumen therethrough; a distal tip at the distal end of the elongated body, wherein the distal tip further includes a distal window; an endoscope positioned through the lumen, wherein an endoscope tip is positioned within the distal window; a vacuum source in fluid communication with the lumen, wherein the vacuum source is configured to draw tissue into the lumen through the distal window; a needle advanceable through the lumen, wherein the needle is configured to be advanced through the tissue, wherein the needle remains within the distal window when advanced through the tissue; and a guidewire advanceable through the needle, the guidewire being configured to advance through the tissue.

In some variations, the techniques described herein relate to a device, further including a handle at the proximal end of the elongated body, wherein the handle includes a vacuum tubing connected to the vacuum source, and wherein the handle includes a guidewire port.

In some variations, the techniques described herein relate to a device, wherein the needle is configured to flex when advanced through the lumen.

In some variations, the techniques described herein relate to a device, further including a needle locking mechanism, wherein the needle locking mechanism is spring-loaded.

In some variations, the techniques described herein relate to a device, wherein the needle is retractable into the elongated body independent from the guidewire.

In some variations, the techniques described herein relate to a device, wherein the distal window is clear.

In some variations, the techniques described herein relate to a device, wherein the distal window includes a radiopaque marker.

In some variations, the techniques described herein relate to a device, wherein the distal window is made of a radiopaque material.

In some variations, the techniques described herein relate to a device, wherein the distal tip further includes a fin configured to break through tissue.

In some variations, the techniques described herein relate to a device, further including a flushing port configured to remove debris from the distal window.

In some variations, the techniques described herein relate to a device, further including a shield to prevent tissue from entering the distal tip.

In some variations, the techniques described herein relate to a device, further including a liner attached to the handle, wherein the liner is configured to shield the guidewire from edges of the needle.

In some variations, the techniques described herein relate to a device for accessing an epicardial space of a patient, the device including: an elongated body including a proximal end, a distal end, and a lumen therethrough; a distal tip at the distal end of the elongated body, wherein the distal tip further includes a distal window; an endoscope positioned through the lumen, wherein an endoscope tip is positioned within the distal window; a vacuum source in fluid communication with the lumen, wherein the vacuum source is configured to draw tissue into the lumen through the distal window; a radiofrequency element advanceable through the lumen, wherein the radiofrequency element is configured to cut through the tissue; and a guidewire advanceable through the radiofrequency element, the guidewire being configured to advance through the tissue.

In some variations, the techniques described herein relate to a method for accessing an epicardial space of a patient, the method including: advancing an elongated body to a target site, the elongated body including a proximal end, a distal end, and a lumen therethrough, wherein a distal tip is positioned at the distal end of the elongated body, the distal tip including a distal window; advancing an endoscope through the lumen, wherein an endoscope tip is positioned within the distal window; drawing a tissue at the target site into the distal window via a vacuum source in fluid communication with the lumen; puncturing the tissue with a needle advanceable through the lumen; advancing a guidewire through a lumen of the needle; and retracting the elongated body, the needle, and the endoscope such that the guidewire remains positioned through the tissue.

### BRIEF DESCRIPTION OF THE DRAWINGS

FIG. 1 illustrates a perspective view of an access device according to one variation of the invention.

FIG. 2 illustrates a method of accessing an epicardial space according to one variation of the invention.

FIG. 3 illustrates an access device with a spring-loaded needle according to another variation of the invention.

FIG. 4 illustrates an access device with a radiofrequency enabled needle according to another variation of the invention.

FIG. 5 illustrates an access device with a roof vent according to another variation of the invention.

FIGS. 6A and 6B illustrate a fixed liner within a needle according to one variation of the device.

FIGS. 7A to 7C illustrate a mechanism for a retracting liner within a needle according to one variation of the device.

FIG. 8 illustrates another variation of the distal tip having a needle bent such that a distal tip of the needle angles down towards an opening of the distal tip.

FIGS. 9A to 9E illustrate a variation of the device having a syringe attached to the handle.

FIGS. 9F and 9G illustrate a cross-sectional view of a needle passing through a reservoir attached to the syringe.

FIGS. 10A and 10B illustrate another variation of the device having a syringe for delivering a contrast agent through the needle.

FIG. 11 illustrates a device having an elongated shaft and one or more navigation electrodes for tracking the location of an end effector at the distal end of the device.

FIGS. 12A to 12C illustrate another variation of a device having a steerable elongated shaft.

FIGS. 13A to 13C illustrate another variation of a device having a steerable elongated shaft.

FIGS. 14A to 14C illustrate another variation of a device having a steerable elongated shaft comprising a wider diameter to accommodate larger diameter electrodes.

FIGS. 15A to 15C illustrate the device of FIGS. 13A to 13C fully deflected as the elongated shaft is positioned near the posterior wall of the heart.

FIGS. 16A to 16C illustrate the device of FIGS. 14A to 14C fully deflected as the elongated shaft is positioned near the posterior wall of the heart.

FIGS. 17A and 17B illustrate the device of FIGS. 13A to 13C in conjunction with an ablation device.

FIGS. 17C and 17D illustrate the device of FIGS. 13A to 13C and the ablation device accessing the heart for a posterior wall ablation.

FIG. 17E illustrates an example of the steering range of the ablation device.

FIGS. 18A and 18B illustrate another variation of a device having an elongated shaft steerable to 90 degrees and a thinner shaft diameter. sss

FIGS. 18C and 18D illustrate the device of FIGS. 18A and 18B accessing the heart for a posterior wall ablation.

FIG. 18E illustrates an example of the steering range of the device of FIGS. 18A and 18B.ss

FIGS. 19A and 19B illustrate the device of FIGS. 14A to 14C in conjunction with an ablation device.

FIGS. 20A to 20D illustrate steerable sheaths according to one variation of the invention.

FIGS. 21A and 21B illustrate a steerable sheath according to another variation of the invention.

### DETAILED DESCRIPTION

Described here are systems, devices, and methods for accessing the epicardial space through the pericardium. While the devices and methods described here are described in reference to puncturing the pericardium to provide access to the heart of a patient, it should be understood that these devices and methods may be used to create a puncture in or otherwise facilitate access to any fluid-filled membrane or sac to access the structures therein, e.g., dura mater, peritoneum, amniotic sac, and the like.

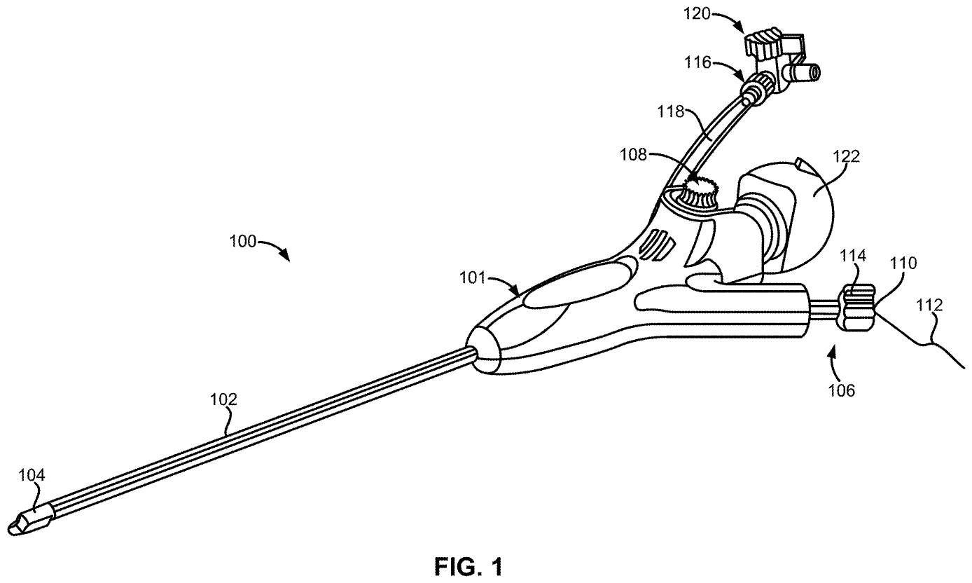

FIG. 1 illustrates a perspective view of an access device 100 according to one variation of the invention. The device 100 can comprise a handle 101, an outer shaft 102, and a distal tip 104 at a distal end of the outer shaft 102.

The handle 101 can comprise a needle actuator 106, a scope actuator 108, and a guidewire port 110 for introducing a guidewire 112 at a proximal end of the handle. The needle actuator 106 can be used to advance or retract a needle 212 through the lumen 206 of the outer shaft 102. In some embodiments, the needle actuator 106 can comprise a piston. The piston can be actuated by a user to control advancement of the needle 212 by proximally or distally moving the piston with respect to the outer shaft 102. Accordingly, the user can control the speed at which the needle 212 is advanced. The needle 212 can thereby be advanced into the distal tip 104 when the distal tip 104 is positioned at a desired location (i.e., at or near the outer pericardium).

In other embodiments, a wheel mechanism can be used to advance and retract the needle 212.

The needle actuator 106 can also be used to control the rotation of the needle 212 within the outer shaft 102. To rotate the needle 212, the user can rotate the piston radially with respect to the outer shaft 102. The piston can comprise grooves or ribs 114 extending axially for the user to grip during rotation of the needle 212.

The needle 212 can flex upward (i.e., away from the pericardium and the epicardium) during advancement of the needle 212. The needle 212 can thus grab tissue and ride up a bleb of the tissue as the needle is advanced to reduce the risk of puncturing the ventricle or surrounding tissue.

The needle actuator 106 can comprise a tab that locks within the handle 101 to ensure orientation of the needle 212 when delivered. This feature provides for safety from the risk of puncturing nearby tissue during advancement of the needle 212 as the needle 212 can maintain the appropriate angle when approaching the tissue. The needle 212 can also have an atraumatic tip or edge that allows the needle 212 to contact tissue without damaging the tissue.

The needle 212 can be a 17 to 25 gauge needle with an atraumatic shape to minimize the risk of dangerous puncture in the event of ventricular stick.

The needle 212 can include a central lumen that extends therethrough, enabling advancement of the guidewire 112 through the device/needle and into the pericardium. The guidewire 112 can be advanced through the needle 212 accordingly when the needle 212 is inserted through a port on the needle actuator 106.

The handle 101 can be coupled to a vacuum port 116 for applying suction through the outer shaft 102. The vacuum port 116 can be connected to a vacuum tubing 118 and a vacuum source (not shown) in fluid communication with the outer shaft 102. A valve 120 can be positioned on the vacuum port 116 to couple the vacuum source with the handle 101. The vacuum source can apply suction to the lumen of the outer shaft 102, which draws the outer pericardium into the distal tip 104 for engagement with the needle 212.

The handle 101 can be coupled to a camera unit 122 which can be coupled to or otherwise embedded within the endoscope 210. The endoscope 210 can be introduced through an endoscope port on the handle 101. The endoscope 210 can comprise a diameter of about 3 mm and can be rigid so as to not interfere with the needle 212 during the procedure but flexible to maneuver as needed through the outer shaft 102. The scope actuator 108 on the handle can rotate and/or axially advance the endoscope 210.

In some embodiments, the handle 101 can be coupled to a flushing port to aid in removing loose tissue and/or debris from the distal tip 104.

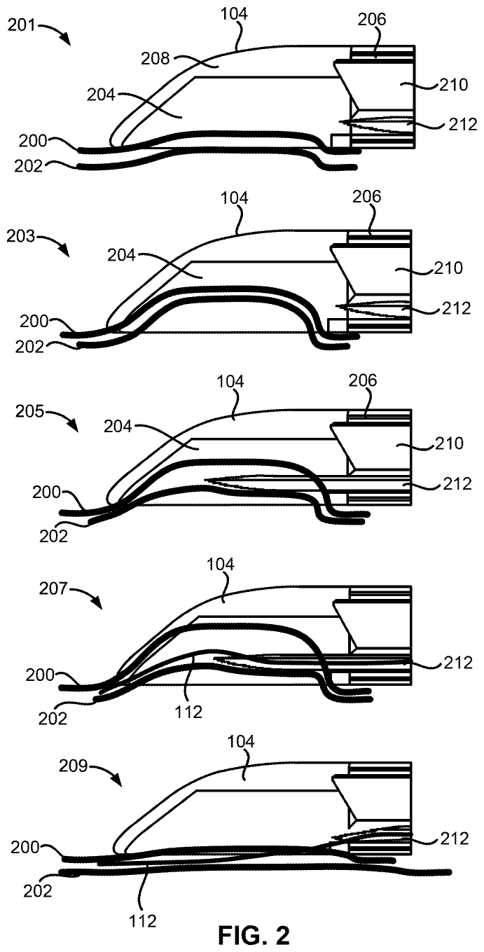

FIG. 2 illustrates a diagram of a method of accessing an epicardial space according to one variation of the invention. The distal tip 104 can be advanced to a location adjacent to a space between the outer pericardium 200 and the epicardium 202 to be accessed. Once the distal tip 104 is in the desired position, the vacuum source can apply a vacuum through the outer shaft to retract the pericardium and the epicardium into a cavity 204 of the distal tip 104.

The outer shaft 102 can house a needle and an endoscope within a lumen 206 of the outer shaft 102. In some embodiments, the outer shaft 102 can comprise a multi-lumen configuration in which the needle and the endoscope can advance within separate lumens. In some embodiments, the outer shaft 102 can comprise an inner shaft disposed within, in which the needle 212 or endoscope 210 can be disposed through while the other of the needle or endoscope is disposed in a space outside of the inner shaft and inside of the outer shaft.

The distal tip 104 of the device 100 can be clear for visualization of the surrounding anatomy during blunt dissection via the endoscope. The distal tip 104 can be radiopaque for the purposes of gross navigation and trajectory control during blunt dissection. The distal tip 104 can comprise a fin on its leading edge to aid in breaking through connective tissue during blunt dissection.

The distal tip 104 can have a clear distal window for visualization. The distal tip 104 can protect a standard endoscope from surrounding tissues enabling dissection and navigation to the pericardium through a sub-xiphoid skin incision under endoscopic visualization. The optically clear window can also include a radiopaque marker or be made from radiopaque material for the user to be able to confirm the distal-most region of the device under fluoroscopy. In some embodiments, the window 208 can have a shield to prevent unwanted tissue from entering the cavity 204 of the distal tip and obstructing the endoscopic view.

The device 100 can enable direct endoscopic visualization of the pericardium before, during, and after entry into the pericardium. The device 100 can be used with commercially available scopes and cameras. The device 100 can also position the endoscope 210 and/or camera with a scope retention mechanism.

Step 201 illustrates the distal tip 104 first delivered to the target location. The endoscope 210 can provide visualization of the target location and the distal tip 104 can be pressed against the heart (i.e., at the outer pericardium and epicardium). Step 203 illustrates when the vacuum source has applied vacuum through the outer shaft 102, resulting in the outer pericardium 200 and the epicardium 202 being retracted into the cavity 204 of the distal tip 104 through the window 208.

Step 205 illustrates the needle 212 advanced into cavity 204 to puncture the outer pericardium 200 and ultimately placed adjacent to the epicardium 202. The endoscope 210 provides visualization of the tissue throughout this process. After the needle has been successfully inserted into the outer pericardium, the guidewire can be advanced through the needle, as seen in step 207. The needle 212 is then removed while the guidewire 112 remains in place as seen in step 209. The device 100 can be removed shortly thereafter if necessary. Since the pericardium 200 is drawn into the device, the needle 212 does not exit the envelope dimensions of the distal window, eliminating the possibility of inadvertent puncture of non-target tissues.

In some embodiments, the clear, blunt dissection window of the distal tip 104 can be a variety of shapes and sizes as well as a variety of textures.

In other embodiments, an integrated chip on the tip system can be used to eliminate the need to insert an endoscope into the device. A video sensor (e.g., CMOS) can be integrated into the distal tip 104 of the device, improving the ergonomics of the system by eliminating the size and weight of the endoscope 210 and camera attached at the proximal end of the embodiment of FIG. 1. This variation can also enable use of a flexible or steerable shaft, and also may reduce the overall crossing profile of the device.

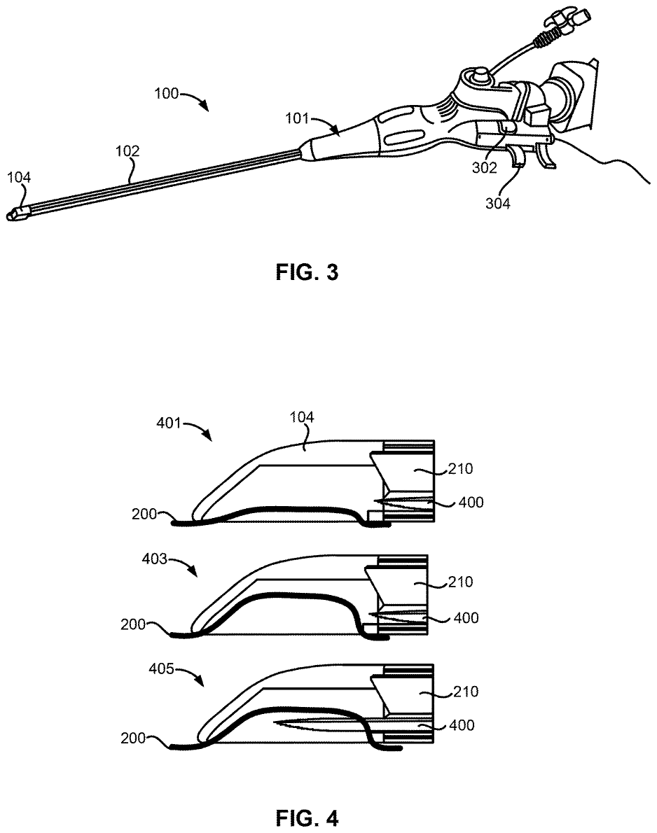

FIG. 3 illustrates an embodiment of an access device with a spring-loaded needle according to another variation of the invention. The spring-loaded needle can be activated by an activation button 302 on the handle 101. The needle 212 can be retracted by a retraction button 304 to reset the needle 212 in a spring-loaded state. This variation can eliminate user influence over advancement of the needle 212 as the activation button 302 and the retraction button 304 can have predetermined limits on how far the needle 212 can move through the outer shaft 102. In some variations, the needle 212 can be a beveled needle, a hypodermic needle, or the like.

FIG. 4 illustrates an access device with a radiofrequency (RF) enabled needle 400 according to another variation of the invention. First, as seen in step 401, the distal tip 104 is delivered to the target site, where it is pressed against the pericardium 200, which enters the window 208. Vacuum is then applied to retract the pericardium further into the window 208, as seen in step 403.

Radiofrequency leads (not shown) can be connected or attached to the needle 400. RF energy can be generated through the radiofrequency leads to gently cut through the pericardium 200 or coagulate as necessary after the pericardium is retracted into the cavity 204, as seen in step 405. The pericardial puncture is not based on axial force, so there is less energy buildup of the tissue and subsequent tissue recoil once the needle 400 punctures through the pericardium 200. Accordingly, there is a low advancement force of the needle 400 during the procedure. Since RF energy does not require significant pressure to gain pericardial puncture, the risk of the energy build up and recoil is reduced.

In some variations, the needle 400 can be a sharp needle, a dull needle, or a blunt hypotube.

In some variations, RF leads can be attached to the guidewire 112 which can cut through pericardium.

In some variations of the device, a smaller needle (about 29 to about 32 Ga) can be introduced through a larger needle (about 22 Ga) to minimize risk during the pericardial puncture. The smaller needle can initially puncture the pericardium. The initial puncture of the smaller needle can inherently reduce the risk of bleeding. Once the initial puncture is made, the vacuum between the pericardium and heart can be broken, allowing air to enter the epicardial space and improving the ability of the vacuum applied to the pericardium to separate the pericardium from the ventricular surface. More separation between the pericardium and ventricular surface greatly reduces the risk of ventricular damage when the second, larger needle is inserted. After the larger needle is inserted, the smaller needle is withdrawn out of the device so that the guidewire can be delivered through the larger needle.

One technical problem encountered by the inventors is separating the pericardium from the ventricle, such that when the needle is advanced through the pericardium, the needle does not stick the ventricle. Technical solutions discovered by the applicants provide for methods of gaining separation between the pericardium and the ventricle prior to advancing the needle through the pericardium that the wire will be fed through.

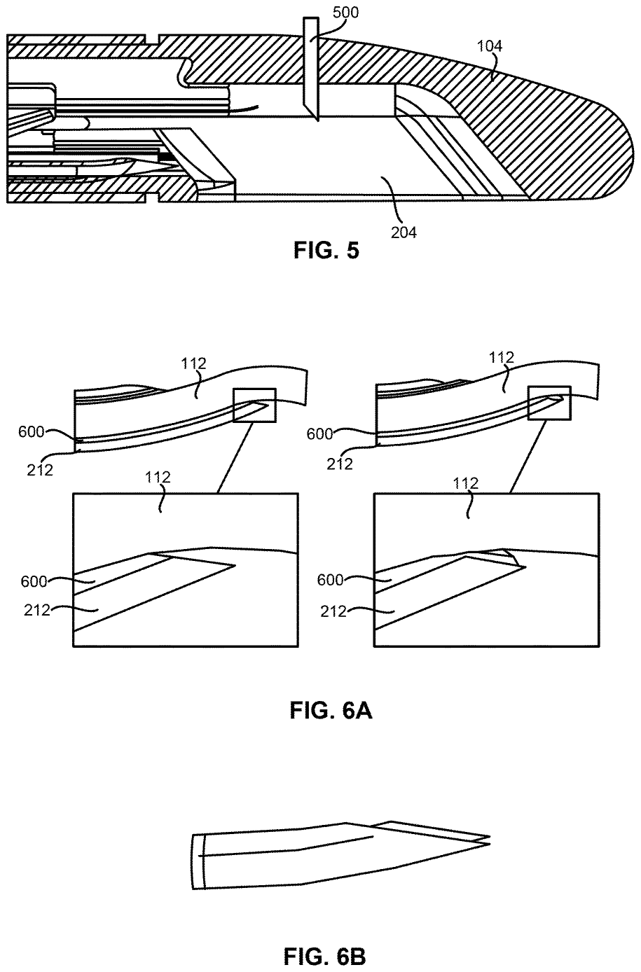

FIG. 5 illustrates one variation of a distal tip with a roof vent for venting or breaking the vacuum state between the pericardium and ventricle to allow the vacuum applied by the device to separate the pericardium from the ventricle or ventricular surface. In this variation, a needle 500 (about 30 Ga) is affixed to the roof of the cavity in the distal tip 104 and communicates outside of the distal tip 104.

As the tissue is retracted into the distal tip 104 via suction, the pericardial tissue raises up into the distal tip 104. The needle 500 in the distal tip roof communicates to the outside of the device and can comprise a lumen such that the vacuum pump can overcome a leak path and still generate enough vacuum to retract the pericardium. When the pericardium retracts into the device, it contacts the needle at the roof of the suction aperture such that the needle punctures the pericardium. The epicardial space is accordingly vented and the pericardium can separate from the ventricle.

One technical problem encountered by the inventors is that advancing, and particularly retracting, a coated guide wire through a needle can result in the sharp internal edges of the distal end of the needle lumen to scrape off the guide wire coating and produce particulate. Technical solutions designed by the applicant comprise design solutions to the needle, which require less prior training for the user when retracting the guidewire.

For example, FIGS. 6A and 6B illustrate a liner 600 within a needle 212 according to one variation of the device. This variation can comprise a thin-walled polymer liner 600 installed inside a needle lumen to protect the guidewire 112 from the inner edges of the needle. The liner can be bonded in place and then laser-trimmed at the distal end to match the grind angle of the needle. The liner may be trimmed flush to the needle 212. In other embodiments, the liner can be trimmed with an intentional overhang from the needle 212 to provide additional protection.

In some embodiments, the liner 600 can be removably coupled to the needle 212.

In some embodiments, a metal hypotube can be used to puncture the pericardial tissue but is not advanced into the epicardial space. Instead, after RF cutting of the tissue, a polymer liner is advanced through the puncture site and into the epicardial space. This embodiment carries less risk of the inadvertently cutting or burning the ventricular tissue with the RF element which is not advanced into the epicardial space. Advancement of the polymer liner can create a lumen into the epicardial space to advance the guidewire 112.

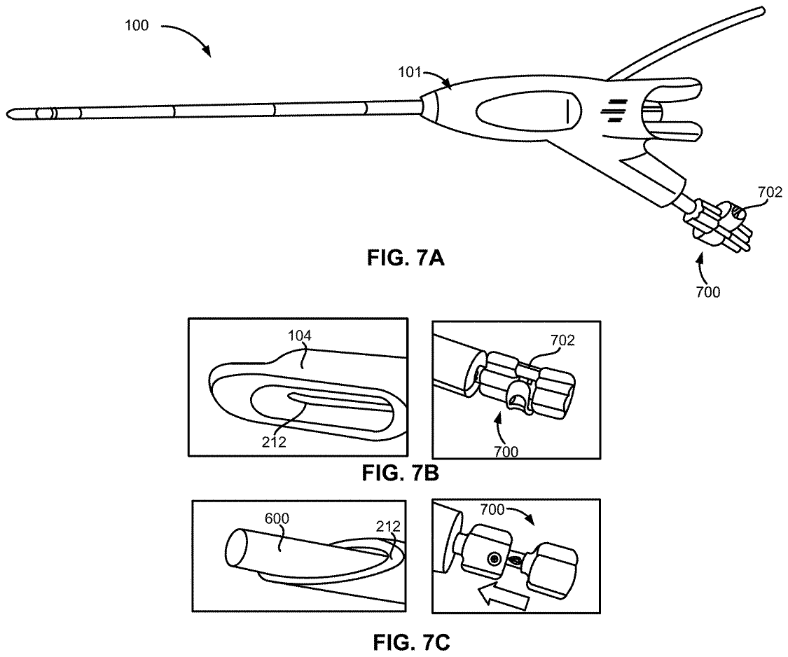

FIGS. 7A to 7C illustrate a mechanism for a retracting liner within a needle according to one variation of the device. In this variation, an extending or retracting liner is attached to a liner actuator 700 at the device handle that allows the user to extend and retract the liner. The liner can be retracted during pericardial puncture so that the liner does not impact needle puncture performance.

A safety clip 702 can be employed along the liner actuator 700 to ensure that the user always extends the liner prior to advancing or retracting the guide wire. The safety clip 702 can clamp onto the liner actuator 700 such that the liner actuator 700 cannot move axially while the needle 212 is positioned within the distal tip 104, as seen in FIG. 7B. Once the safety clip 702 is removed, the liner can then be extended once the needle is in the epicardial space to provide ample protection for the guide wire, as seen in FIG. 7C.

In some embodiments, a laser process can be used to melt an inner sharp edge of the distal tip of the needle 212 to produce a smooth radius to prevent damage to the guidewire 112 from moving past the edge of the needle 212.

In some embodiments, the needle can have an adhesive applied to an inner edge thereof. In this variation, an adhesive can be applied to the inner edge of the needle to create a radius and reduce the risk of guide wire damage. In some embodiments, the adhesive can be ultraviolet-cured.

In some embodiments, the needle can be made with a deep cut during fabrication. The cut could be made by EDM, laser, waterjet, or milling to reduce the sharp edge formed by the needle manufacturing process.

In other embodiments, an abrasive slurry process could be used which involves flowing an abrasive slurry through the needle to soften the internal edge.

In other embodiments, a diamond wire grinding process can be used for fabrication of the needle. The process uses a diamond wire file to grind the opening of the needle to a smooth configuration. Accordingly, this reduces the risk of the needle shaving off particulate from the guidewire when the guidewire is passed through.

The diamond grinding process can comprise a machine for spinning the diamond wire in order to grind the inside of the needle. The diamond wire can be rotated while the needle is held by a platform that oscillates the needle back and forth. A heel attack angle, a toe attack angle, and a nominal angle all can be used for which the needle is ground to during the procedure.

A fixture setup for the grinding process can comprise a computer unit with a configurator connected to a control box. The control box can be coupled to the fixture mechanism via a wire. The control box can also be coupled to a wire connector for uploading a grinding program as desired by the user.

After grinding, the interior of the needle can have a smooth surface such that a guidewire can pass through with less risk of particulate being shaved off into the anatomy.

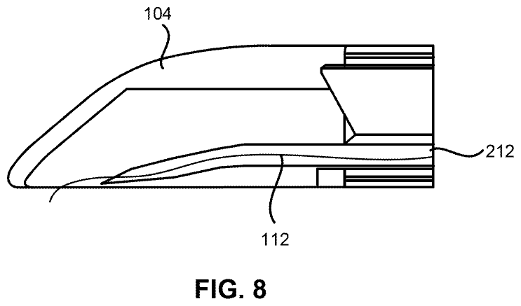

FIG. 8 illustrates another variation of the distal tip 104 having a needle 212 bent such that a distal tip of the needle angles down towards an opening of the distal tip 104 for smooth guidewire 112 access against the tissue after penetration of the needle 212. This configuration can change the needle 212 access angle toward the pericardium, allowing for easier needle penetration of fat or fibrous tissue on the pericardium surface. The bent configuration of the needle 212 can also serve to direct the needle 212 away from the ventricular surface after penetrating the pericardium, reducing the risk of inadvertent ventricular stick.

In some variations, the bend angle of the needle 212 can be predetermined and fixed during manufacture. In other variations, the bend angle can be adjustable by the user, allowing the clinician to customize the needle trajectory based on patient anatomy or the thickness of pericardial fat encountered.

The bent needle configuration can further facilitate advancement of the guidewire 112 into the epicardial space at a shallow angle relative to the tissue surface, reducing the risk of the guidewire 112 buckling or redirecting upon entry into the pericardial space. As with other needle variations described herein, the needle 212 in this configuration can comprise a lumen through which the guidewire 112 is advanced following successful pericardial puncture, and the needle 212 can include a liner 600 or smoothed inner edge to protect the guidewire coating during advancement and retraction through the needle lumen.

One technical problem encountered by the inventors is how to achieve needle penetration and visualization with increased vacuum pressure when there is fat on the pericardium. One technical solution discovered by the inventors is to provide a high force to advance guidewire after needle penetration with high vacuum pressure to enable needle penetration with fat on pericardium. This also provides a smoother guidewire advancement after penetration, limiting the challenges of impeded direct visualization with fat, which prevent the confirmation of correct needle penetration.

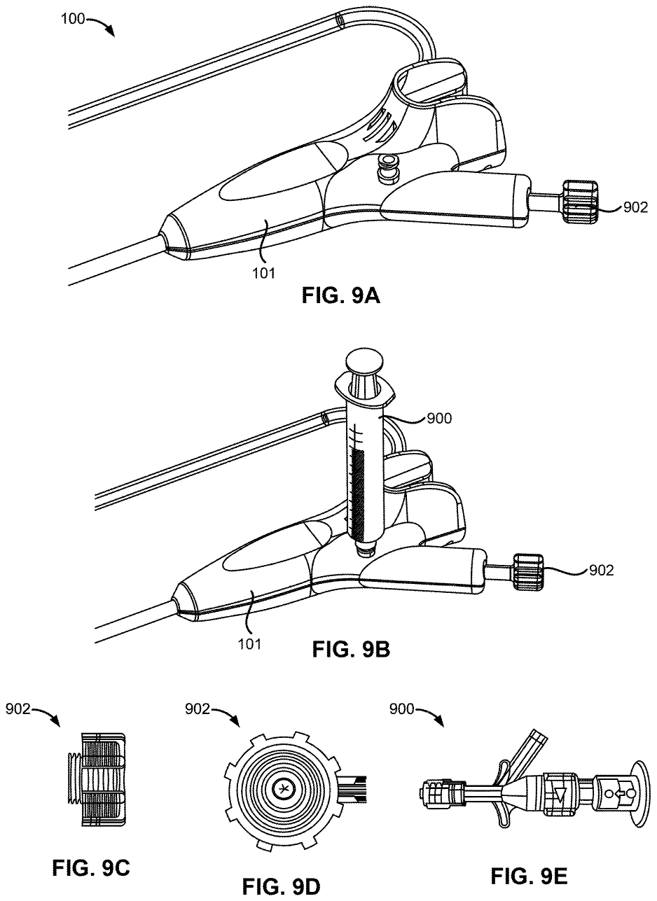

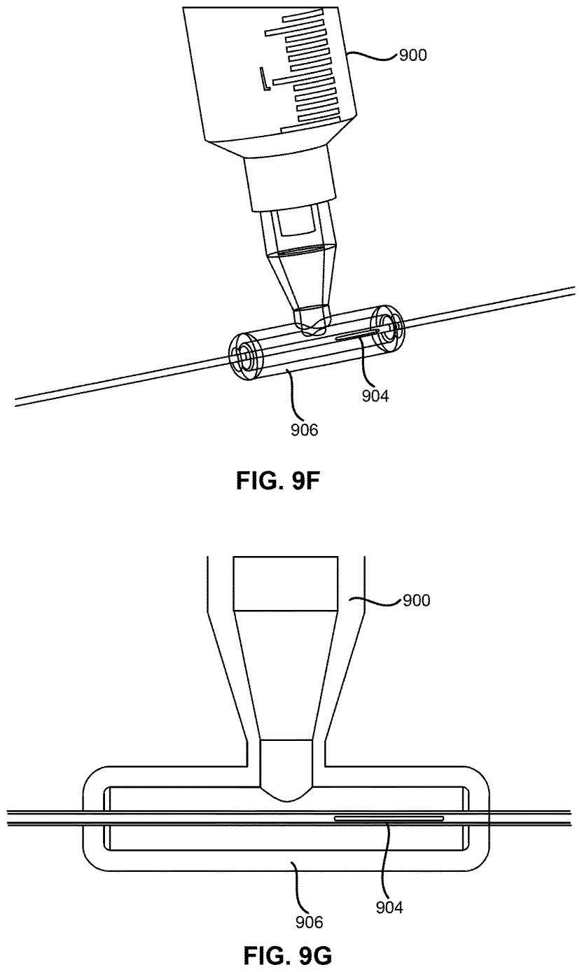

FIGS. 9A to 9E illustrate a variation of the device 100 having a syringe 900 attached to the handle 101. The syringe 900 can be fluidly coupled to the needle 212 through the handle 101 to allow a user to aspirate or inject fluid through the needle lumen during the procedure. The device 100 can also comprise a hemostasis valve 902 for controlling access to the needle lumen and for creating a fluid-tight seal around any instrument or guidewire passed therethrough, preventing air embolism during the procedure. The hemostasis valve 902 can be located at a Y-connector configured to provide simultaneous access to the guidewire 112, allowing the guidewire 112 to be advanced through the hemostasis valve 902 and into the needle lumen without requiring removal of the syringe 900 or interruption of the fluid-tight seal.

In some variations, the hemostasis valve 902 can comprise a compression fitting. In some variations, aspiration of fluid through the syringe 900 prior to guidewire 112 advancement can be used to confirm needle placement within the pericardial space, as the return of pericardial fluid or the absence of blood can provide the clinician with confirmation that the needle 212 has successfully traversed the pericardium without inadvertent ventricular puncture. The syringe 900 can further be used to introduce saline or other biocompatible fluid to hydrodissect the pericardial tissue from the epicardium, augmenting the separation achieved by the vacuum source and the venting mechanisms described herein.

FIGS. 9F and 9G illustrate a cross-sectional view of a needle passing through a reservoir 906 attached to the syringe 900. The reservoir can be located within the handle 101, interposed between the syringe 900 and the proximal end of the needle 904. The reservoir 906 can be configured to maintain a small volume of fluid in fluid communication with the needle lumen, such that any fluid return upon successful pericardial entry is immediately visible to the user within the reservoir 906 without requiring the user to aspirate actively.

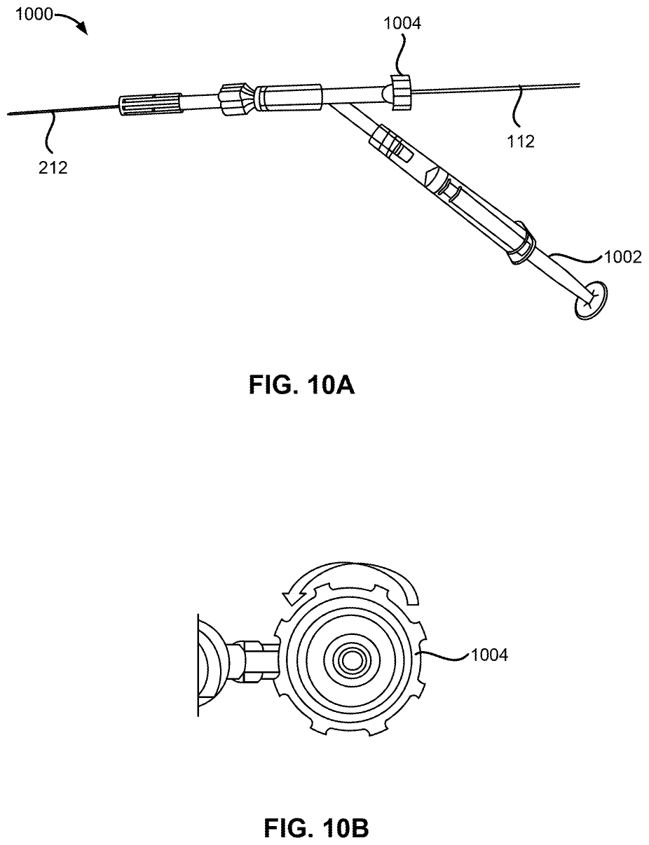

FIGS. 10A and 10B illustrate another variation of the device 1000 having a syringe 1002 for delivering a contrast agent through the needle 212. The syringe 1002 can be coupled to the handle 101 in a manner similar to the syringe 900 described with respect to FIGS. 9A through 9G, and can similarly incorporate a hemostasis valve or Y-connector to permit simultaneous guidewire access. Delivery of a contrast agent through the needle 212 can provide the capability to confirm correct needle penetration into the pericardial space under fluoroscopic visualization, enabling the clinician to observe the distribution of contrast within the epicardial space in real time before advancing the guidewire 112. The characteristic spreading pattern of contrast within the pericardial space can provide a reliable fluoroscopic indicator of correct placement.

In some variations, the syringe 1002 can be preloaded with a predetermined volume of contrast agent sufficient for visualization without risk of over-distension of the pericardial space. The syringe 1002 can further incorporate a one-way valve or backflow limiter to prevent pericardial fluid from passively refluxing into the syringe 1002 during cardiac systole, maintaining the integrity of the contrast delivery system throughout the procedure. In some variations, the contrast delivery capability of the syringe 1002 can be used in combination with the endoscopic visualization provided by the endoscope 210 or the integrated chip-on-tip sensor described herein, providing the clinician with both direct visual and fluoroscopic confirmation of correct pericardial access.

The device 1000 can further comprise a male luer 1004 rotatable to release the guidewire 112. The male luer 1004 can be configured to selectively engage and disengage the guidewire 112 through rotational motion, allowing the clinician to lock the guidewire 112 in a fixed axial position during needle advancement and contrast delivery, and subsequently release the guidewire 112 for advancement into the pericardial space once correct placement has been confirmed. In some variations, the male luer 1004 can threadably engage a corresponding female luer fitting on the handle 101, such that rotation in a first direction tightens a compression seal against the guidewire 112 and rotation in an opposite direction relaxes the seal to permit guidewire translation. The male luer 1004 can be sized and configured for single-hand operation, enabling the clinician to release and advance the guidewire 112 without repositioning the hand holding the device 1000.

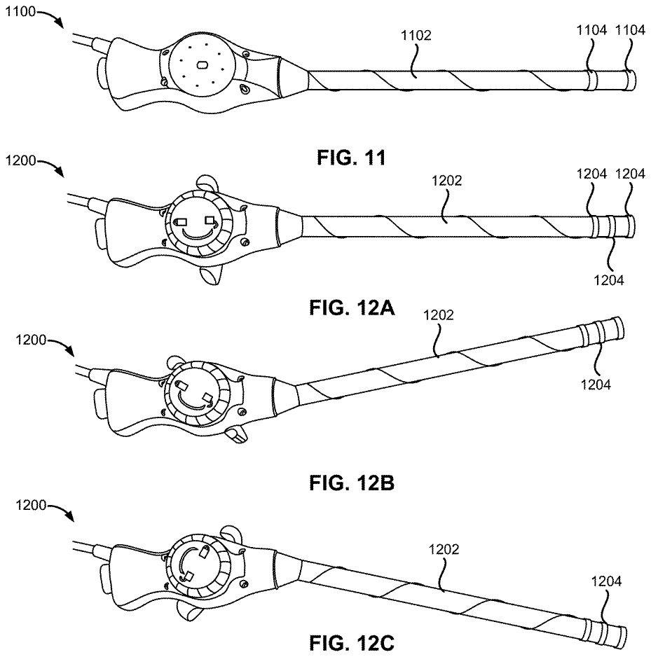

FIG. 11 illustrates a device 1100 having an elongated shaft 1102 and one or more navigation electrodes 1104 for tracking the location of an end effector at the distal end of the device. This variation comprises a fixed elongated shaft 1102 which does not provide for steering or articulation of the distal end in any direction. The navigation electrodes 1104 can be configured to communicate with an external mapping or navigation system to provide real-time positional feedback during advancement of the device through the epicardial space. The navigation electrodes 1104 can be positioned at or near the distal end of the elongated shaft 1102 to provide accurate localization of the end effector relative to cardiac anatomy.

In some variations, the navigation electrodes 1104 can be used in conjunction with fluoroscopic guidance or electroanatomic mapping systems to confirm proper positioning of the device prior to ablation or other therapeutic interventions. The fixed elongated shaft 1102 can provide a stable, predictable trajectory during advancement, which may be advantageous in anatomical regions where a consistent approach angle is desired. In some variations, the elongated shaft 1102 can be sized and configured to be introduced through a sub-xiphoid incision, consistent with the epicardial access approach described herein.

FIGS. 12A to 12C illustrate one variation of a device 1200 having a steerable elongated shaft 1202. The elongated shaft 1202 can be steerable or articulated at the base of the elongated shaft 1202 via controls on the handle. The elongated shaft 1202 can be flexible over its length to allow for bending throughout. The device 1200 can comprise a camera integrated at a distal end of the elongated shaft 1202 such that the device 1200 can steer the elongated shaft 1202 while providing visualization for the user. The integrated camera at the distal end can be a CMOS or similar video sensor, as described with respect to other embodiments herein, providing direct visualization of the epicardial surface and surrounding anatomy during navigation and therapeutic delivery.

The steerable elongated shaft 1202 can allow the user to navigate the device 1200 to anatomical locations that would be inaccessible with a fixed shaft, including regions of the posterior wall of the left atrium and other complex cardiac surfaces. The steering controls on the handle can comprise one or more actuators, such as pull wires, rack-and-pinion mechanisms, or rotatable knobs, configured to deflect the distal portion of the elongated shaft 1202 in one or more planes.

In some variations, the elongated shaft 1202 can be deflectable through a range of angles sufficient to reach the entirety of the epicardial surface from a sub-xiphoid approach. In some variations, the device 1200 can further comprise navigation electrodes similar to those described with respect to device 1100, enabling simultaneous electroanatomic tracking and visualization during the procedure.

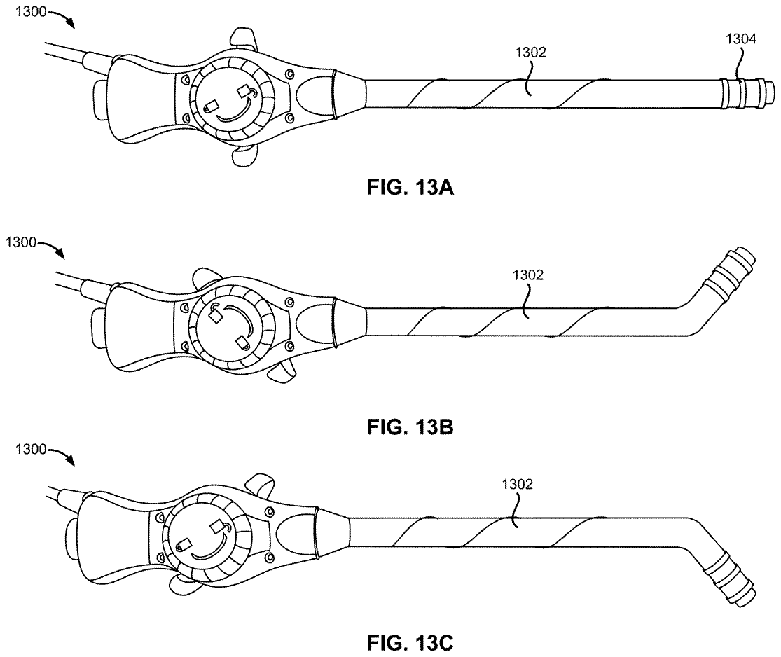

FIGS. 13A to 13C illustrate one variation of a device 1300 having a steerable elongated shaft 1302. The elongated shaft 1302 can be steerable or articulated at a sharp angle towards the distal end of the elongated shaft 1302 via controls on the handle. The remainder of the elongated shaft 1302 can be rigid. The sharp distal deflection of the elongated shaft 1302 can enable the device 1300 to redirect the end effector and electrodes 1304 at or near the distal tip toward target tissue regions, such as the posterior wall of the left atrium, while the proximal rigid portion of the elongated shaft 1302 maintains a stable, low-profile trajectory through the sub-xiphoid access corridor. The steerable distal segment can be actuated by one or more pull wires coupled to controls on the handle, allowing the user to precisely control the deflection angle and direction.

In some variations, the deflection angle of the steerable distal segment can range from about 0 degrees to about 180 degrees, enabling access to a broad range of epicardial targets from a single insertion point. The rigid proximal portion of the elongated shaft 1302 can provide column strength to facilitate advancement and torque transmission through the access corridor without unintended bending or buckling. In some variations, the elongated shaft 1302 can comprise a working channel through which ablation devices, guidewires, or other instruments can be advanced to the target site while the shaft remains in a deflected configuration.

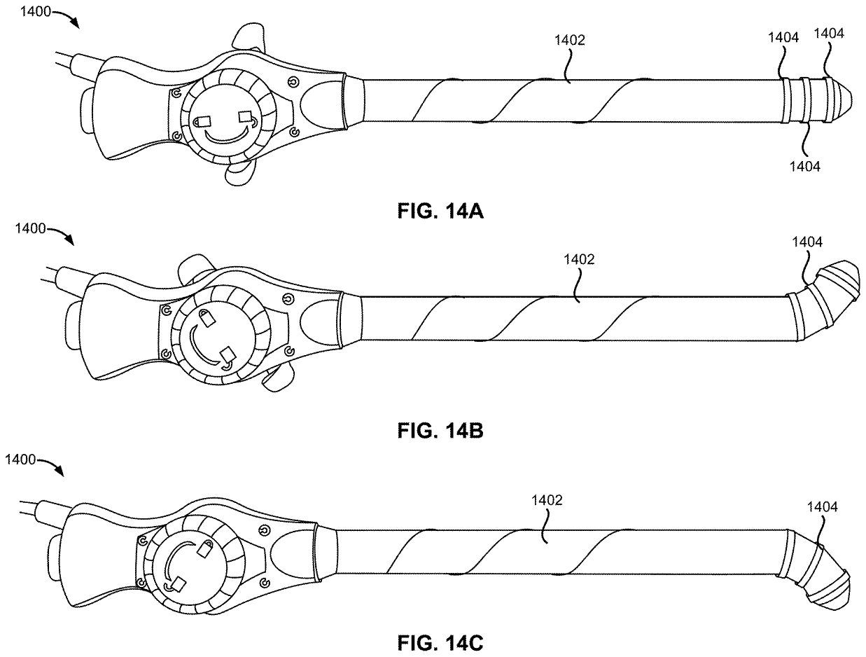

FIGS. 14A to 14C illustrate one variation of a device 1400 having a steerable elongated shaft 1402. The elongated shaft 1402 can be steerable or articulated at a sharp angle towards the distal end of the elongated shaft 1402 via controls on the handle. The remainder of the elongated shaft 1402 can be rigid. In this variation, the elongated shaft 1402 can comprise a wider diameter to accommodate larger diameter electrodes 1404. The wider diameter of the elongated shaft 1402 can provide a larger working channel lumen, enabling the passage of ablation electrodes or other therapeutic devices with a greater cross-sectional profile than those accommodated by the elongated shaft 1302 of device 1300. The larger diameter electrodes 1404 can be configured to deliver radiofrequency energy, cryoenergy, or other ablative modalities over a greater tissue contact area, which may be advantageous when creating longer or broader lesion sets on the epicardial surface.

In some variations, the wider shaft diameter can also accommodate multi-electrode arrays or catheters designed for both mapping and ablation, enabling a combined diagnostic and therapeutic workflow within a single device insertion. The steerable distal segment of the elongated shaft 1402 can be actuated in a manner similar to that described with respect to device 1300, with pull wire or equivalent mechanisms coupled to handle controls allowing precise directional deflection. In some variations, the increased shaft diameter can further provide enhanced torque response, facilitating rotational positioning of the electrodes 1404 against target tissue.

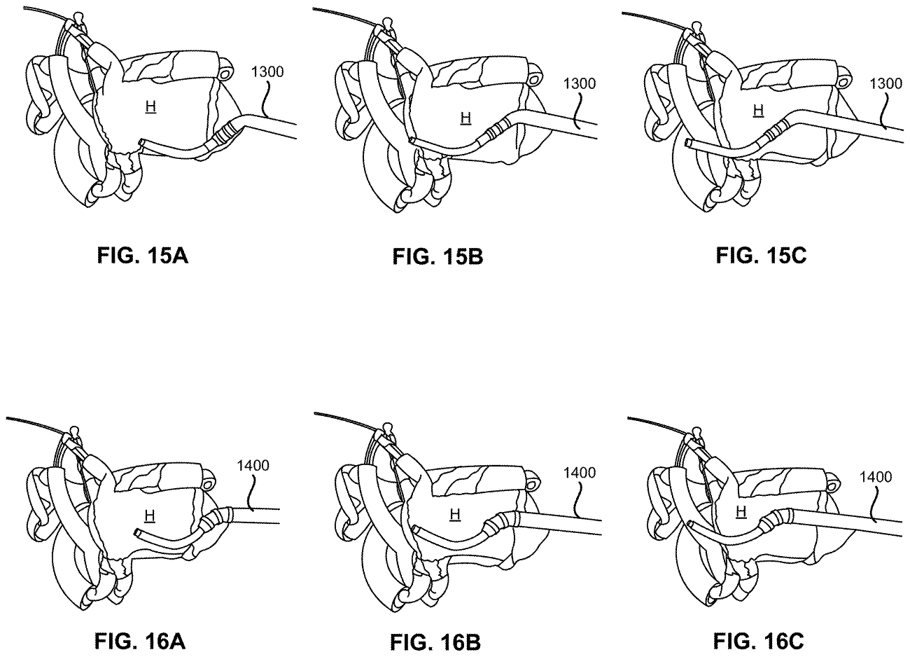

FIGS. 15A to 15C illustrate the device 1300 fully deflected as the elongated shaft 1302 is positioned near the posterior wall of the heart H. FIG. 15A illustrates the device 1300 accessing the apex of the heart, FIG. 15B illustrates the device 1300 being advanced to the middle of the posterior wall, and FIG. 15C illustrates the device 1300 accessing the middle of the posterior wall. As shown in these figures, the sharp distal deflection of the elongated shaft 1302 enables the device 1300 to redirect from the sub-xiphoid insertion axis toward the posterior cardiac surface, which lies in a substantially different anatomical plane. The ability of the device 1300 to access the apex, mid-posterior wall, and other regions of the posterior left atrium from a single entry point is enhanced by the utility of the steerable shaft configuration for comprehensive epicardial coverage.

In some variations, the user can adjust the deflection angle of the elongated shaft 1302 during the procedure to transition between anatomical targets, such as progressing from the apex to the posterior wall, without requiring repositioning of the proximal access corridor. The sequential positions illustrated in FIGS. 15A to 15C reflect a representative navigation path that can be used to systematically map or ablate the epicardial surface of the posterior left atrium, useful for treatment of atrial fibrillation and other cardiac arrhythmias.

FIGS. 16A to 16C illustrate the device 1400 fully deflected as the elongated shaft 1402 is positioned near the posterior wall of the heart H. FIG. 16A illustrates the device 1400 accessing the apex of the heart, FIG. 16B illustrates the device 1400 being advanced to the middle of the posterior wall, and FIG. 16C illustrates the device 1400 accessing the middle of the posterior wall. The navigation path illustrated in FIGS. 16A to 16C is similar to that described with respect to device 1300 in FIGS. 15A to 15C, with the device 1400 similarly capable of transitioning between apical and posterior wall positions through adjustment of the steerable distal segment of the elongated shaft 1402. The wider diameter of the elongated shaft 1402 and the larger diameter electrodes 1404 accommodated thereby can provide enhanced tissue contact and energy delivery at each of the positions illustrated, which can result in more durable or transmural lesions compared to smaller electrode configurations.

In some variations, the device 1400 can be used to create a continuous or contiguous lesion set across the posterior wall by sequentially positioning the electrodes 1404 at adjacent tissue locations as the elongated shaft 1402 is advanced from the apex toward the middle and superior portions of the posterior wall.

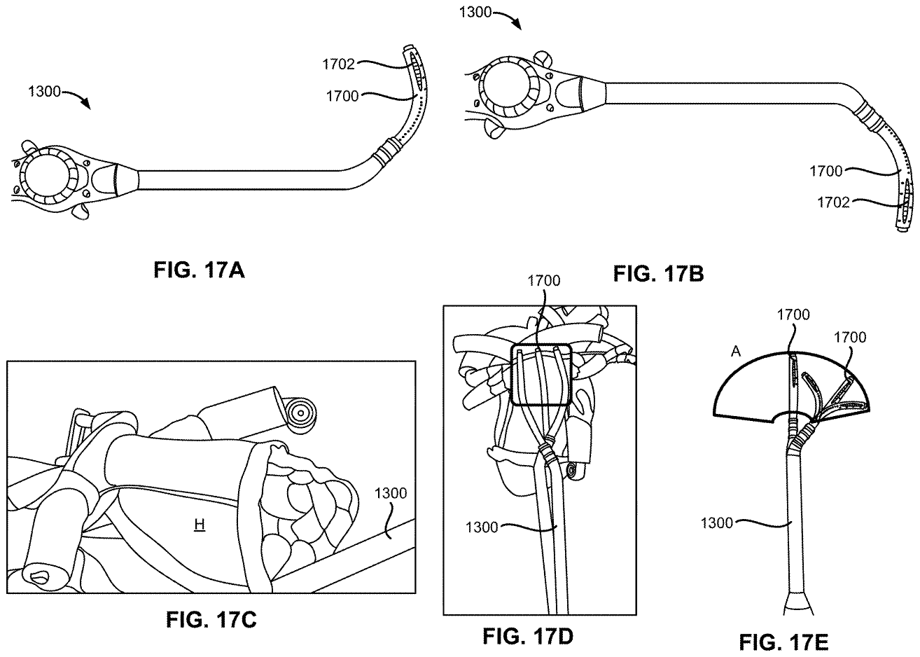

FIGS. 17A and 17B illustrate device 1300 in conjunction with an ablation device 1700. The ablation device 1700 can be inserted through device 1300 and can apply radiofrequency energy to create lesions on the exterior of the heart. The ablation device 1700 can bend at the same angle(s) as device 1300. FIGS. 17C and 17D illustrate the device 1300 and ablation device 1700 accessing the heart for a posterior wall ablation, with FIG. 17D illustrating various rotational positions. FIG. 17E illustrates an example of the steering range of the device 1700. The ablation device 1700 can be advanced through the working channel of the elongated shaft 1302 such that the distal end of the ablation device 1700 emerges at or near the distal tip of the device 1300, enabling the delivery of RF energy to epicardial tissue under the guidance and positioning control provided by the steerable shaft 1302. The conformance of the ablation device 1700 to the deflection angle of the elongated shaft 1302 can ensure that the ablation electrode 1702 at the distal end of the device 1700 is directed toward the target tissue surface in a predictable and controlled manner.

In some variations, the ablation device 1700 can comprise one or more ablation electrodes 1702, thermocouples, or contact force sensors at its distal end to optimize energy delivery and confirm adequate tissue contact during lesion formation. The steering range illustrated in FIG. 17E can represent the full range of deflection via angle A achievable by the device 1700 as it exits the distal tip of the elongated shaft 1302, reflecting the combined maneuverability provided by the steerable shaft and the ablation device together. In some variations, the ablation device 1700 can be used to perform posterior wall isolation or other lesion sets relevant to the treatment of persistent or long-standing persistent atrial fibrillation via an epicardial approach.

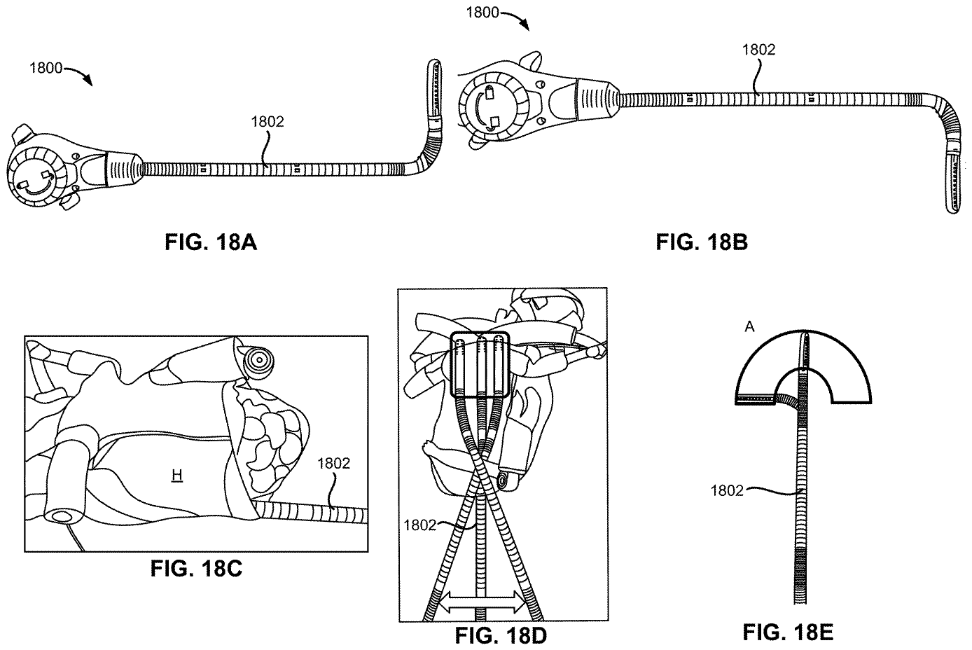

FIGS. 18A and 18B illustrate another variation of a device 1800 having an elongated shaft 1802 steerable to 90 degrees and a thinner shaft diameter. FIGS. 18C and 18D illustrate the device 1800 accessing the heart for a posterior wall ablation. FIG. 18E illustrates an example of the steering range of the device 1800. The 90-degree steering capability of the elongated shaft 1802 can enable the device 1800 to redirect the distal end from the sub-xiphoid insertion axis to a substantially perpendicular orientation relative to the shaft, which may be particularly advantageous for accessing the posterior wall of the left atrium and other anatomical targets that lie at a large angle from the entry trajectory. The thinner shaft diameter of the device 1800 relative to devices 1300 and 1400 can reduce the overall crossing profile of the device, facilitating introduction through smaller incisions or access corridors and potentially reducing patient trauma.

In some variations, the thinner shaft diameter can also provide greater flexibility during navigation within the epicardial space, allowing the device 1800 to conform more readily to the curvature of the cardiac surface. The steering range illustrated in FIG. 18E can reflect the full range of angular positions achievable by the steerable distal segment of the elongated shaft 1802, demonstrating the device's capability to address a broad range of posterior wall anatomies from a single sub-xiphoid insertion point. In some embodiments, the device 1800 can be used in combination with an ablation device or other therapeutic instrument advanced through its working channel, similar to the configurations described with respect to devices 1300 and 1400.

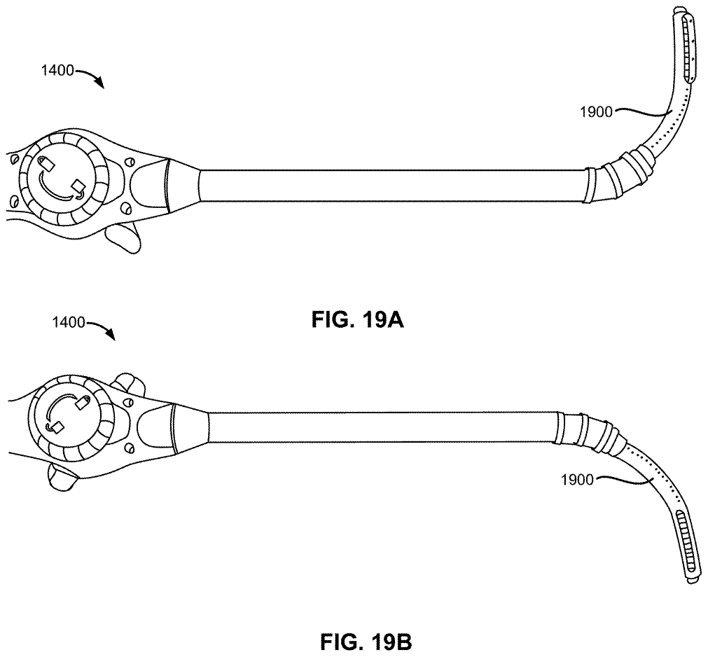

FIGS. 19A and 19B illustrate the device 1400 in conjunction with an ablation device 1900. The ablation device 1900 can be inserted through device 1400 and can apply radiofrequency energy to create lesions on the exterior of the heart. The ablation device 1900 can bend at the same angle(s) as device 1400. The ablation device 1900 can be advanced through the wider working channel lumen of the elongated shaft 1402, enabling the use of larger diameter ablation electrodes that can deliver RF energy over a greater tissue contact area than those accommodated by the elongated shaft 1302 of device 1300. The combination of the wide-lumen elongated shaft 1402 and the larger ablation device 1900 can be particularly suited for procedures requiring robust, transmural lesion sets, such as posterior wall isolation in the treatment of persistent atrial fibrillation.

In some variations, the ablation device 1900 can comprise a multi-electrode array, a linear ablation catheter, or other electrode configuration suited for creating extended lesion sets on the epicardial surface of the posterior left atrium. The conformance of the ablation device 1900 to the deflection angle of the steerable distal segment of the elongated shaft 1402 can ensure accurate and reproducible positioning of the ablation electrode against target tissue as the device 1400 is navigated to successive positions along the posterior wall.

In some variations, the ablation device 1900 can further comprise one or more sensors, such as thermocouples, impedance monitors, or contact force sensors, configured to provide real-time feedback on tissue contact and energy delivery to optimize lesion formation and reduce the risk of inadequate or excessive ablation.

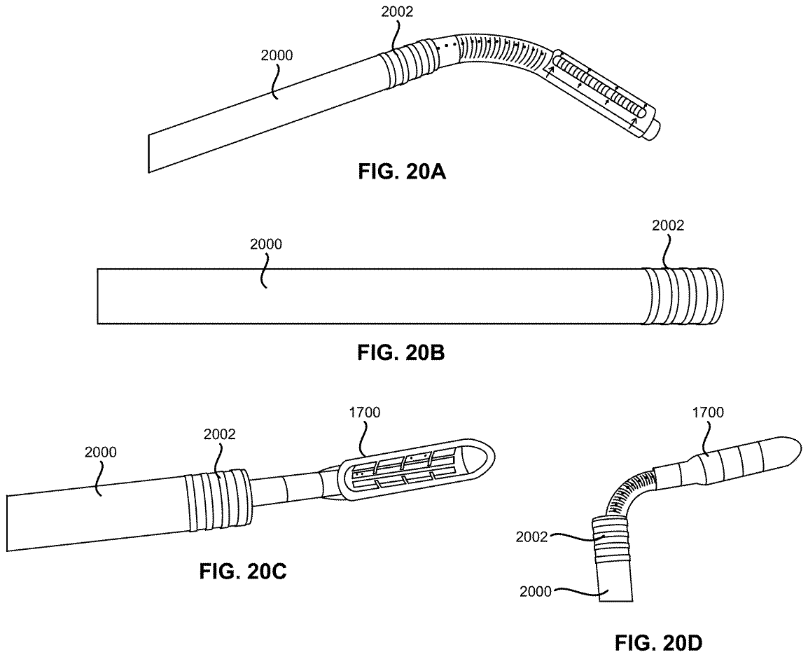

FIGS. 20A to 20D illustrate steerable sheaths according to one variation of the invention. The sheath 2000 can comprise one or more conductive rings 2002 disposed around the full or partial circumference of the distal end of the sheath 2000. Each conductive ring 2002 can be electrically isolated from the adjacent rings 2002 such that the rings 2002 can be individually addressed. The conductive rings 2002 can be coupled to an external mapping system to enable pacing and/or sensing functionality, visibility, and/or positional tracking of the sheath 2000 within the anatomy. In some variations, the conductive rings 2002 can additionally enable physical or electroanatomic mapping via the sheath 2000 itself. The inner and outer diameter of the sheath 2000 can be fixed. In some variations, the sheath 2000 can be manufactured in multiple sizes to ensure compatibility with the corresponding device introduced therethrough. The sheath 2000 can comprise a fixed-curve or straight configuration.

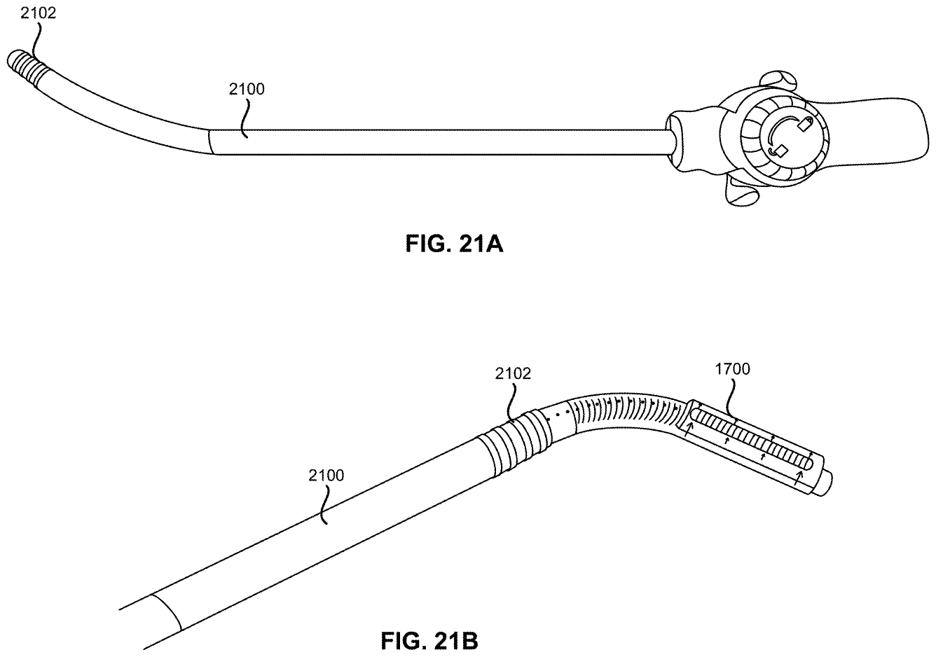

FIGS. 21A and 21B illustrate a steerable sheath 2100 according to another variation of the invention. The sheath 2100 can be bidirectionally or unidirectionally steerable and can comprise one or more conductive rings 2102 disposed around the full or partial circumference of the distal end effector of the sheath 2100. The inner and outer diameter of the sheath 2100 can be fixed. Each conductive ring 2102 can be electrically isolated from the adjacent rings 2102 such that the rings 2102 can be individually addressed. The conductive rings 2102 can be coupled to an external mapping system to enable pacing and/or sensing functionality, visibility, and/or positional tracking of the sheath 2100 within the anatomy.

In some variations, the conductive rings 2102 can additionally enable physical or electroanatomic mapping via the sheath 2100 itself. In some variations, the sheath 2100 can be manufactured in multiple sizes to ensure compatibility with the corresponding device introduced therethrough. In some variations, the steerable sheath 2100, used as a system with an instrument introduced therethrough, can enable articulation in at least two different planes, providing enhanced maneuverability for accessing complex anatomical targets.

A number of embodiments have been described. Nevertheless, it will be understood by one of ordinary skill in the art that various changes and modifications can be made to this disclosure without departing from the spirit and scope of the embodiments. Elements of systems, devices, apparatus, and methods shown with any embodiment are exemplary for the specific embodiment and can be used in combination or otherwise on other embodiments within this disclosure. For example, the steps of any methods depicted in the figures or described in this disclosure do not require the particular order or sequential order shown or described to achieve the desired results. In addition, other steps or operations may be provided, or steps or operations may be eliminated or omitted from the described methods or processes to achieve the desired results. Moreover, any components or parts of any apparatus or systems described in this disclosure or depicted in the figures may be removed, eliminated, or omitted to achieve the desired results. In addition, certain components or parts of the systems, devices, or apparatus shown or described herein have been omitted for the sake of succinctness and clarity.

Accordingly, other embodiments are within the scope of the following claims and the specification and/or drawings may be regarded in an illustrative rather than a restrictive sense.

Each of the individual variations or embodiments described and illustrated herein has discrete components and features which may be readily separated from or combined with the features of any of the other variations or embodiments. Modifications may be made to adapt a particular situation, material, composition of matter, process, process act(s) or step(s) to the objective(s), spirit, or scope of the present invention.

Methods recited herein may be carried out in any order of the recited events that is logically possible, as well as the recited order of events. Moreover, additional steps or operations may be provided or steps or operations may be eliminated to achieve the desired result.

Furthermore, where a range of values is provided, every intervening value between the upper and lower limit of that range and any other stated or intervening value in that stated range is encompassed within the invention. Also, any optional feature of the inventive variations described may be set forth and claimed independently, or in combination with any one or more of the features described herein. For example, a description of a range from 1 to 5 should be considered to have disclosed subranges such as from 1 to 3, from 1 to 4, from 2 to 4, from 2 to 5, from 3 to 5, etc. as well as individual numbers within that range, for example 1.5, 2.5, etc. and any whole or partial increments therebetween.

All existing subject matter mentioned herein (e.g., publications, patents, patent applications) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise, all technical and scientific terms used herein have the same meaning as commonly understood by one of ordinary skill in the art to which this invention belongs.

Reference to the phrase “at least one of”, when such phrase modifies a plurality of items or components (or an enumerated list of items or components) means any combination of one or more of those items or components. For example, the phrase “at least one of A, B, and C” means: (i) A; (ii) B; (iii) C; (iv) A, B, and C; (v) A and B; (vi) B and C; or (vii) A and C.

In understanding the scope of the present disclosure, the term “comprising” and its derivatives, as used herein, are intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Also, the terms “part,” “section,” “portion,” “member” “element,” or “component” when used in the singular can have the dual meaning of a single part or a plurality of parts. As used herein, the following directional terms “forward, rearward, above, downward, vertical, horizontal, below, transverse, laterally, and vertically” as well as any other similar directional terms refer to those positions of a device or piece of equipment or those directions of the device or piece of equipment being translated or moved.

Finally, terms of degree such as “substantially”, “about” and “approximately” as used herein mean the specified value or the specified value and a reasonable amount of deviation from the specified value (e.g., a deviation of up to ±0.1%, ±1%, ±5%, or ±10%, as such variations are appropriate) such that the end result is not significantly or materially changed. For example, “about 1.0 cm” can be interpreted to mean “1.0 cm” or between “0.9 cm and 1.1 cm.” When terms of degree such as “about” or “approximately” are used to refer to numbers or values that are part of a range, the term can be used to modify both the minimum and maximum numbers or values.

It will be understood by one of ordinary skill in the art that the various methods disclosed herein may be embodied in a non-transitory readable medium, machine-readable medium, and/or a machine accessible medium comprising instructions compatible, readable, and/or executable by a processor or server processor of a machine, device, or computing device. The structures and modules in the figures may be shown as distinct and communicating with only a few specific structures and not others. The structures may be merged with each other, may perform overlapping functions, and may communicate with other structures not shown to be connected in the figures. Accordingly, the specification and/or drawings may be regarded in an illustrative rather than a restrictive sense.

This disclosure is not intended to be limited to the scope of the particular forms set forth, but is intended to cover alternatives, modifications, and equivalents of the variations or embodiments described herein. Further, the scope of the disclosure fully encompasses other variations or embodiments that may become obvious to those skilled in the art in view of this disclosure.

We claim:

1. A device for accessing an epicardial space of a patient, the device comprising:
an elongated body comprising a proximal end, a distal end, and a lumen therethrough;
a distal tip at the distal end of the elongated body, wherein the distal tip further comprises a distal window;
an endoscope positioned through the lumen, wherein an endoscope tip is positioned within the distal window;
a vacuum source in fluid communication with the lumen, wherein the vacuum source is configured to draw tissue into the lumen through the distal window;
a needle advanceable through the lumen, wherein the needle is configured to be advanced through the tissue, wherein the needle remains within the distal window when advanced through the tissue; and
a guidewire advanceable through the needle, the guidewire being configured to advance through the tissue.

2. The device of claim 1, further comprising a handle at the proximal end of the elongated body, wherein the handle comprises a vacuum tubing connected to the vacuum source, and wherein the handle comprises a guidewire port.

3. The device of claim 1, wherein the needle is configured to flex when advanced through the lumen.

4. The device of claim 1, further comprising a needle locking mechanism, wherein the needle locking mechanism is spring-loaded.

5. The device of claim 1, wherein the needle is retractable into the elongated body independent from the guidewire.

6. The device of claim 1, wherein the distal window is clear.

7. The device of claim 6, wherein the distal window comprises a radiopaque marker.

8. The device of claim 1, wherein the distal window is made of a radiopaque material.

9. The device of claim 1, wherein the distal tip further comprises a fin configured to break through tissue.

10. The device of claim 1, further comprising a flushing port configured to remove debris from the distal window.

11. The device of claim 1, further comprising a shield to prevent tissue from entering the distal tip.

12. The device of claim 2, further comprising a liner attached to the handle, wherein the liner is configured to shield the guidewire from edges of the needle.

13. A device for accessing an epicardial space of a patient, the device comprising:
an elongated body comprising a proximal end, a distal end, and a lumen therethrough;
a distal tip at the distal end of the elongated body, wherein the distal tip further comprises a distal window;
an endoscope positioned through the lumen, wherein an endoscope tip is positioned within the distal window;
a vacuum source in fluid communication with the lumen, wherein the vacuum source is configured to draw tissue into the lumen through the distal window;
a radiofrequency element advanceable through the lumen, wherein the radiofrequency element is configured to cut through the tissue; and
a guidewire advanceable through the radiofrequency element, the guidewire being configured to advance through the tissue.

14. A method for accessing an epicardial space of a patient, the method comprising:
advancing an elongated body to a target site, the elongated body comprising a proximal end, a distal end, and a lumen therethrough, wherein a distal tip is positioned at the distal end of the elongated body, the distal tip comprising a distal window;
advancing an endoscope through the lumen, wherein an endoscope tip is positioned within the distal window;
drawing a tissue at the target site into the distal window via a vacuum source in fluid communication with the lumen;
puncturing the tissue with a needle advanceable through the lumen;
advancing a guidewire through a lumen of the needle; and
retracting the elongated body, the needle, and the endoscope such that the guidewire remains positioned through the tissue.
